(19) United States
(12) Patent Application Publication
(10) Pub. No.: US 2004/0087920 A1
(43) Pub. Date: May 6, 2004

(54) Ostomy device kit

(76) Inventor: Robert W. Etheredge III, Natick, MA (US)

Correspondence Address: Alvin Isaacs, Esq., 9544 Hawksmoor Lane, Sarasota, FL, 34238-3221, US

(21) Appl. No.: 10/693,582

(22) Filed: Oct. 27, 2003

Related U.S. Application Data
(60) Provisional application No. 60/421,558, filed on Oct. 28, 2002.

(51) Int. Cl. A61F 5/44

(52) U.S. Cl. 604/332

(57) ABSTRACT

An ostomy product for use by ostomates for collecting bodily waste products following a colostomy, ileosotomy or urostomy, comprising a dispenser container having therein a roll of ostomy pouches, the adjacent pouches in the roll being separated by a fracture line for easy separation of the lead pouch from the roll, each of the pouches comprising two opposed sheets of a flexible and expandable liquid-impermeable material secured together along their respective edges to provide a liquid-impermeable enclosure defining an expandable chamber for collecting and retaining bodily waste materials from the ostomate, the pouch having fracture lines substantially centrally disposed between two opposed edges of one of the sheets defining an opening which is revealed when the fracture lines are broken to separate the material between the fracture lines, thereby providing an entry port for bodily waste products exiting from the ostomate's stoma.

### RELATED APPLICATION

This (non-provisional) patent application replaces my provisional patent application Serial No. 60/421,558 filed Oct. 28, 2002.

### BACKGROUND OF THE INVENTION

This invention relates to novel ostomy device kits and, more particularly to novel ostomy device kits which are low cost alternatives to the ostomy pouches which are currently on the market from a number of manufacturers, including Holster, ConvaTec (B. M. Squibb), Coloplast, etc.

Current Medicare allowances for ostomy appliances are insufficient for many ostomates who are then faced with the choice of either paying for these supplies out-of-pocket or washing and then reusing their supplies. This latter alternative inherently exposes the user to potentially unclean conditions, including the risk of infection from e. coli bacteria.

As mentioned above, the present invention is directed to an alternative to the current commercially available products, such as those mentioned above, albeit without their technical features. Specifically, one novel feature of the present invention upon which patentable novelty is herein predicated is its reliance on a novel commodity plastic construction and facile converting by a manufacturer as will be described in detail hereinafter.

The ostomy device kits of this invention permit more frequent pouch appliance changes by the user. In fact, at anticipated costs for this device, the pouches are so cost-effective that they may be replaced as often as desired. This feature in turn significantly reduces if not obviating the need for add-on features such as multilayer plastics, activated charcoal filters and liquid/solid waste handling designs currently found in substantially more expensive pouches presently on the market.

Yet another important feature of the present invention is that the ostomy device kits of the invention can readily be adapted to mate with many if not all of the currently available pouch systems, thus providing a potentially much wider market for sale to ostomates.

For these reasons, it should thus be readily apparent from the following detailed description that the present invention will provide an inexpensive supply of ostomy kits which also obviate the need for reuse.

### BRIEF DESCRIPTION OF THE INVENTION

The novel ostomy kits of this invention comprise a roll of ostomy pouches disposed in a dispenser box much like trash bags. The roll of pouches are sealed on all four sides, with the side between adjacent pouches preferably scored or perforated to permit easy dispensing. As will be appreciated, the edge seals for each pouch must be of sufficient width and strength to retain the waste contents without rupturing.

Each of the individual pouches are provided with attachment means to be described in detail hereinafter with reference to the accompanying drawings.

### BRIEF DESCRIPTION OF THE DRAWINGS

FIG. 1 is a plan view illustrating a typical ostomy skin barrier wafer with flange of the prior art adhesively fastened over the stoma port of an ostomate;

FIG. 2 is a perspective view of the typical prior art ostomy bag or pouch (Applicant believes the term “pouch” to be more common, although either is correct) with a molded flange adapted to mate with the flange of the ostomy skin barrier wafer shown ijn FIG. 1, the bag having an open end opposed from the bag opening and molded flange;

FIG. 3 is a perspective view illustrating positioning of an attachment flange on a pouch in preparation of securing the pouch to the skin of an ostomate via the attachment flange;

FIG. 4 is a plan view illustrating two adjacent pouches of the roll;

FIG. 5 is a schematic view showing how the individual pouches may be applied to the skin surrounding the stoma port;

FIG. 6 is a perspective view of the attachment flange of FIG. 5

FIG. 7 is a plan view of the pouch first in the “open” position, showing sealing means in the form of adhesive strips for sealing the bag after use; and

FIG. 8 is a diagrammatic view illustrating a preferred embodiment in the assembly of the barrier flange, pouch and release liner of each individual ostomy device of the invention.

### DETAILED DESCRIPTION OF THE INVENTION

As was mentioned earlier, the present invention is directed to a low cost alternative to the known ostomy pouches currently commercially available.

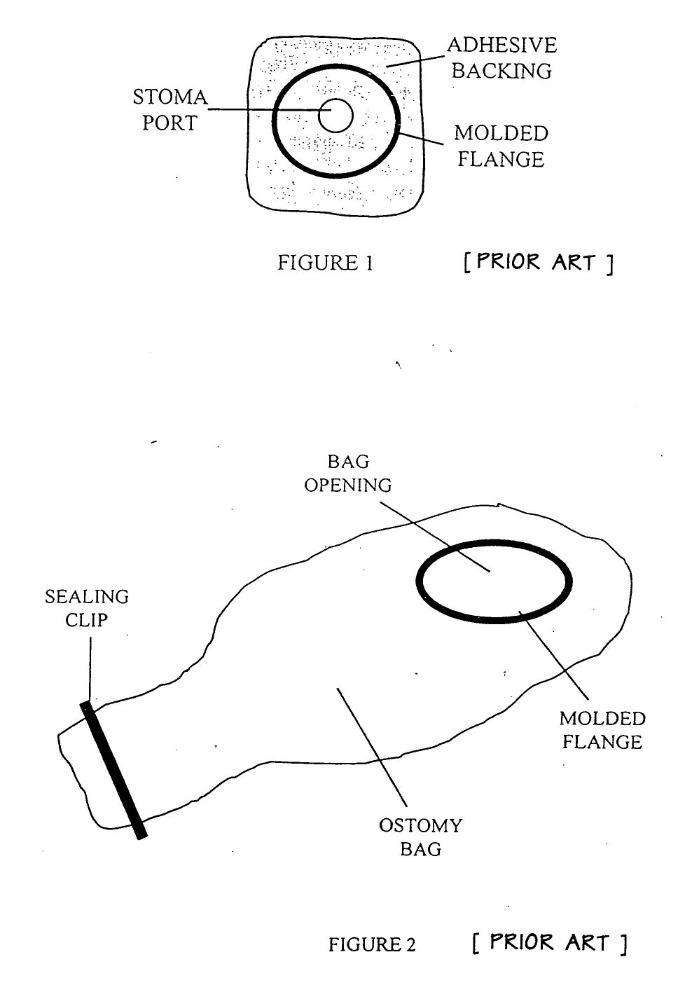

The ostomy appliances of the prior art, as illustrated in FIGS. 1 and 2 will generally comprise two basic parts. The first part (illustrated in FIG. 1) is called an “ostomy skin barrier” or “skin barrier wafer”. While shown to be generally rectangular, the shape is not important and it may, for example, be generally circular or ovate. In any case, as manufactured by the aforementioned suppliers, it will comprise an adhesive substrate, e.g. a dermatologically acceptable pressure-sensitive adhesive for adhering the flange to the skin surrounding the stoma port of the ostomy.

The second part (as shown in FIG. 2) is a pouch or bag that attaches to the molded flange of the skin barrier shown in FIG. 1 by means of a mating molded flange on the pouch. When mated, together they make a snap-together, liquid-tight seal. The pouch functions to catch bodily waste products from the stoma following a colostomy, ileostomy or urostomy surgical procedure ; while the adhesive layer of the ostomy skin barrier serves to retain the combination in place on the ostomate.

As will be apparent, the pouch must have removable closure means for maintaining the liquid-tight seal to prevent accidental or unwanted premature emptying or leakage of any of the body waste products from within the pouch. In FIG. 2, a sealing clip, a preferred form of sealing the trailing end of the pouch, is illustrated as the closure means.

When full or otherwise in need of replacement, the pouch is unsnapped, after which the sealing clip may be released for emptying the pouch of the bodily waste products contained therein.

The bag, with its mating flange, may then be disposed of or reused, depending upon its style and contemplated usage. In any case, each manufacturer employs its own flange design, so that barrier flanges and pouches from different manufacturers do not mate, thus precluding purchasing them from different manufacturing sources.

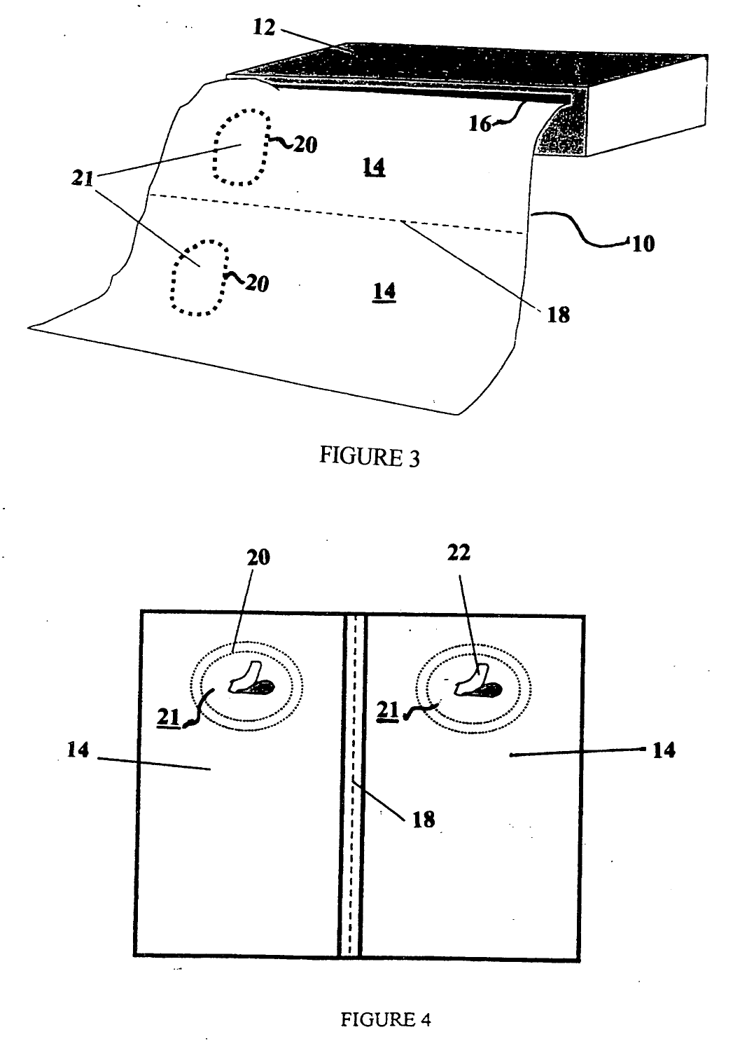

The present invention will now be readily understood by reference to the following detailed description taken in conjunction with the accompanying illustrative drawings of FIGS. 3-8.

With reference to FIGS. 3 and 4, the present invention contemplates a roll 10 having a plurality of individual ostomy pouches 14 separated from one another by fracture or score lines 18 for easy separation for individual use. As seen in FIG. 3, the roll of ostomy bags 10 may be packaged for sale in a per se known dispenser box 12 much like that which has been used for trash bags. The dispenser box 12 is illustrated in FIG. 3 to be provided with a side opening 16 for withdrawing the leading end of the roll of pouches 10. The individual pouches are sealed on all four sides adjacent to one another, with adjacent pouches separated by score lines 18, as heretofore noted, for ease of separation.

The pouches 10, which of course will be fluid-impervious, may be made from any of the known cost-effective materials, e.g., low density polyethylene (LDPE), high density polyethylene (HDPE), polyvinyl chloride (PVC) or other commodity plastics as well as biodegradable versions. In any case, the selection of the particular fluid-impervious material to be employed per se comprises no part of this invention and will therefore be a matter of individual choice within the expected knowledge of the skilled worker, especially in the light or this detailed description of the invention.

The individual pouches may be sealed in any of the per se known manners, heat-stamping being a particularly useful manner from a manufacturing standpoint. The perimeter edge seal between pouches should be of sufficient strength against rupturing to retain the waste product contents. To this end, the perimeter edge seal between adjacent pouches should be relatively wide, e.g. on the order of from about 0.5 to about 1.0 inch in width.

Each of the pouches from the roll as shown in FIG. 3 may be used in combination with an ostomy skin barrier wafer provided with a barrier flange 36 similar to those of the prior art as previously discussed and illustrated in FIG. 1. The barrier flange may be a component of the package containing the roll of pouches, or they may be packaged and sold separately, as are the two prior art components.

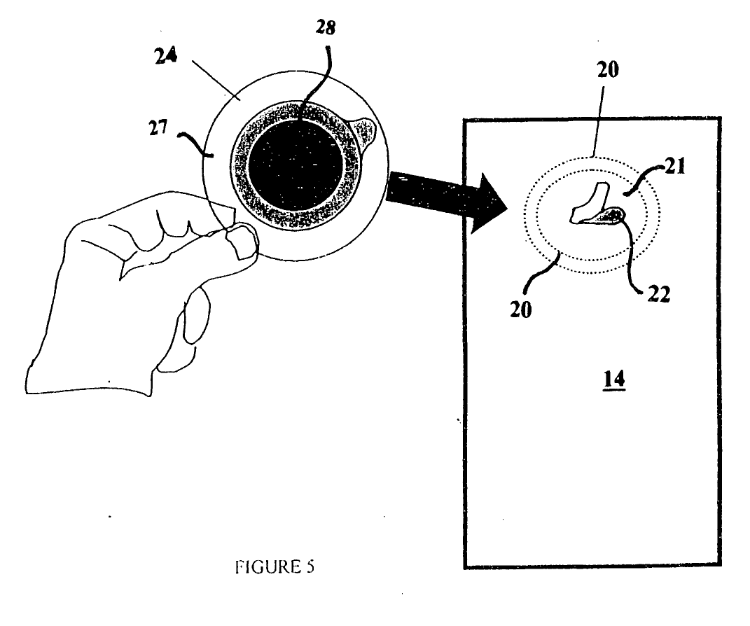

Each of the pouches in the roll of this invention will most preferably, but not essentially, be provided with score or fracture lines 20 as illustrated in FIGS. 4 and 5 to define a window 21 open to the chamber within the pouch when the fracture lines are broken to release the pouch material therebetween, heretofore termed a “window” to the pouch chamber the window is preferably generally circular, as illustrated in the drawings.

In the preferred form, a plurality of concentric rings of fracture lines will be provided to accommodate differences in individual ostomy dimensions. Although two concentric rings are shown in the accompanying drawings, more or less are within the scope of this invention.

In any case, a removal tab 22 is also provided to facilitate lifting of the severed window for clean separation from the pouch.

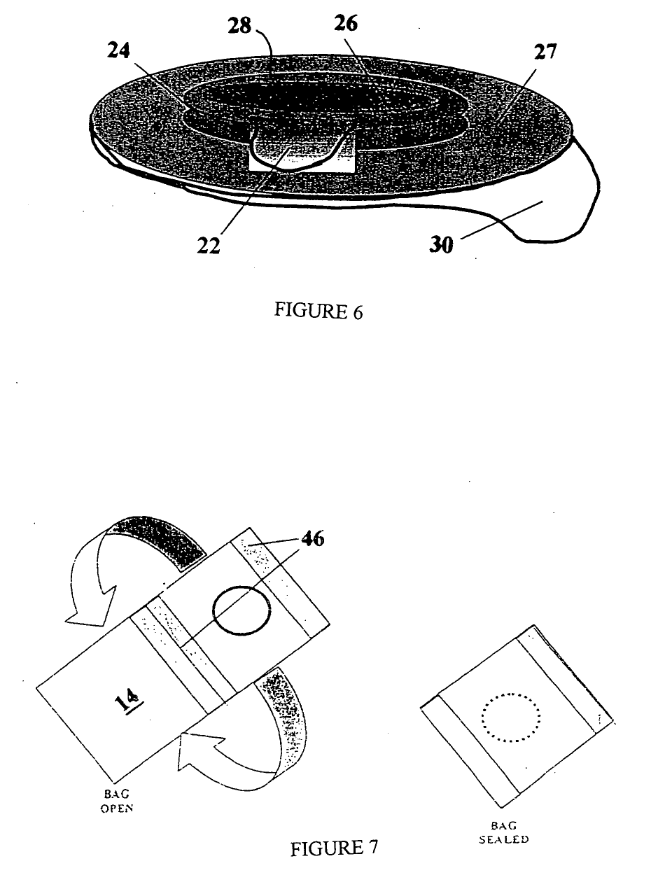

With reference to FIGS. 5 and 6, the attachment flange 24 for attaching the pouch to the skin barrier wafer, like the pouches previously described, may be manufactured from LDPE, HDPE, PVC, or other commodity plastics or biodegradable materials, as were the pouches. While its configuration is not critical from a functional standpoint, however for aesthetic reasons it will be substantially of a circular configuration, as shown in the drawings. In any case, it may be so manufactured by injection molding.

As best seen in FIG. 6, the bag attachment flange 24 has a central portion 26 which is generally circular and which extends outwardly away from the base portion 27 terminate in a central opening 28 which is of substantially the same size and configuration as the window opening 21 on the pouches to be applied over the stoma in usage. In the illustrative embodiment shown in the drawings, since this opening 21 in the pouch is substantially circular, the open end of the pouch attachment flange 24 will likewise be of substantially the same size and dimensions.

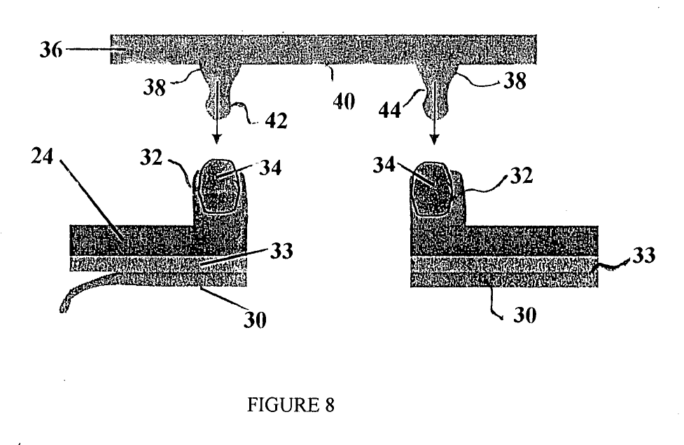

The base portion 27 of the pouch attachment flange is generally planar and is coated with a layer 33 (as seen in FIG. 8) of any of the per se known adhesives, preferably a pressure-sensitive adhesive sufficiently aggressive to adhere the attachment means to the plastic surfaces to which adherence of the article is contemplated. The selection of a suitable adhesive to use will be a matter of choice within the expected purview of the manufacturer and as such per se comprises no part of this invention. However, for purposes of a full and complete disclosure, for application to polyethylene surfaces such as the aforementioned LDPE or HDPE, the selected adhesive may be a tackified, rubber-based adhesive commericially available from various sources or an acrylic pressure-sensitive adhesive with high adhesion to such nonpolar substrates. Similar conventional adhesives are of course commercially available for use with other plastics that may be contemplated in the practice of this invention. As seen in FIG. 6, a per se known release liner 30 protects the attachment flange from unwanted premature contact with surfaces to which it might adhere. On removal of the release sheet, The attachment means is molded with a second side, i.e. the opposed side from that to be pressed and secured to the pouch, which second side contains an adhesive coating for affixing it to an ostomate skin barrier wafer as previously described and illustrated in FIG. 1 and which, in turn, has previously been adhesively secured to the skin of the ostomate user.

While the prior art ostomy supplies, as was previously mentioned in general require the pouch which mates with the barrier layer be provided from the same supplier, an important innovative feature of the present invention is the ability of the attachment flange secured to the pouch to mate with any of the barrier flanges currently commercially available over-the-counter.

In one embodiment, as illustrated in FIG. 8, the opposed side of the attachment flange 24 from the adhesive-coated side thereof for attachment of the flange to the pouch, contains a channel 32 containing a soft adhesive or non-adhesive polymer 34 employed to form a shape complementary to that of a flange 36 carried on the ostomy skin barrier layer 35.

For example, a polyisobutylene or ethylene vinyl acetate (EVA) polymer or equivalent with appropriate additives as known in the art and which then requires no further description may be employed.

Thus, when the polymer is pre-softened, e.g. by means of conventional household items such as a hair dryer or a microwave oven, it will form a mating shape complementary to the barrier layer when the pouch containing the attachment flange having the softened polymer in the channel is pressed against he barrier layer and then allowed to harden by allowed it to cool.

As illustrated in FIG. 8, flange 36 may have irregularly shaped projections 38 extending from the base 40 of the flange to its tip 42. As seen, the projections taper inwardly from the base 38 to a median point 44 and then commence to taper outwardly towards the tip 42 of the projection. While the polymer 32 in the channel 34 of the pouch flange 24 is still soft and moldable, the pouch 14 is pressed firmly against the surface of the barrier layer 35 so that the projections 38 are pressed into the channel 32 of the pouch flange 24. When cooled to harden, the polymer 32 will then conform to the irregular configuration of the projections 38 of barrier flange 36, more of the polymer being in the median point 44 to insure a locking engagement attaching the pouch to the ostomate

In still another embodiment, the flange itself is made of a softenable polymer that will mold to a complementary and mating form when heated and then cooling as it hardens. The polymers chosen for this purpose must soften only above the anticipated maximum shipping temperature (e.g. 140° F.), but below the melting point of the pouch material, e.g. the melting point of polyethylene which is ˜230° F.

In yet another embodiment, the attachment means may be molded in a variety of profiles matching each of the pouches employed. Users would then simply purchase a box of the appropriate size attachment means which will then be applied to the pouches of this invention. A range of sizes is anticipated to account for differences in individual ostomy dimensions. The pouches may accordingly have several concentric rings of scoring lines to accommodate different size orifices to be made.

When ready to be applied, the release liner covering the adhesive layer is removed and then the molded part of the of the attachment means is pressed against the pouch in the designated target area as shown in FIG. 5, and the detachable “window” is removed.

FIG. 7 illustrates a means for sealing the pouch for disposal after it is removed from the ostomate. As shown therein, pouch 14 has a pair of adhesive strips 46 each of which is initially covered with a release sheet (not shown). The pouch is initially present in the open position, as shown in the left hand illustration. Following removal, the release sheet(s) may be removed and the pouch folded over, as shown in the illustration, to provide a sealed bag ready for disposal.

While the pouch is shown for purposes of illustration with two sealing strips, it is within the scope of the invention to provide more or less sealing strips.

Since various changes may be made without departing from the scope of the invention herein contemplated and defined in the appended claims, it is intended that the foregoing description and the accompanying drawings shall be interpreted as illustrative only and not in a limiting sense.

What is claimed is:

1. An ostomy product for use by ostomates for collecting bodily waste products following a colostomy, ileostomy, or urostomy, comprising a dispenser container having therein a roll of ostomy pouches, the adjacent pouches in the roll being separated by a fracture line to facilitate easy separation of the lead pouch in the roll from the other pouches in the roll, each of the pouches having two opposed flexible sheets of a flexible liquid-impermeable material sealed together along their respective edges to provide a liquid-impermeable enclosure defining an expandable chamber for collecting and retaining bodily waste materials from the ostomate; the pouch having fracture lines substantially centrally disposed between two opposed edges of one of the sheets defining an opening which is revealed when the fracture lines are broken to separate the material between the fracture lines, thereby permitting the bodily waste products to pass through the stoma port to within the pouch chamber.

2. An ostomy product as defined in claim 1 including a barrier flange sheet material having an adhesive coating on one side thereof for securing the barrier sheet material, the barrier flange material having a substantially centrally disposed opening defining a stoma port for egress of bodily waste products from the stoma; the pouch being releasably secured to the barrier flange with the opening of the sheet of the pouch in alignment with the stoma port in the barrier sheet, whereby bodily waste products emanating from the stoma port will collect through this opening into the cavity of the pouch defined by the pouch walls.

3. The method for recovering bodily waste products from an ostomate following a colostomy, ileostomy or urostomy surgical procedure by securing an ostomy pouch for collecting the bodily waste products to an ostomate's skin to receive he bodily waste products discharged from the ostomy;
the improvement wherein the ostomy pouch is a pouch as defined in claim 1.

4. In the method for receiving and collecting bodily waste products from an ostomate following a colostomy, ileostomy or urostomy surgical procedure by securing an ostomy pouch for collecting the bodily waste products to the ostomate's skin to receive the bodily waste products discharged from the ostomy:
the improvement wherein the ostomy pouch is a pouch as defined in claim 2.
